(19) United States
(12) Patent Application Publication
(10) Pub. No.: US 2009/0259095 A1
(43) Pub. Date: Oct. 15, 2009

(54) System and method for treatment of hypo-orgasmia and anorgasmia

(76) Inventors: Nicholas Sieveking, Nashville, TN (US); Michael Stephanides, Nashville, TN (US); Carl Zimmerman, Nashville, TN (US)

Correspondence Address: BRINKS HOFER GILSON & LIONE, P.O. BOX 10395, CHICAGO, IL, 60610, US

(21) Appl. No.: 12/291,603

(22) Filed: Nov. 12, 2008

Related U.S. Application Data
(60) Provisional application No. 60/987,216, filed on Nov. 12, 2007.

(51) Int. Cl. A61N 1/36

(52) U.S. Cl. 600/38; 607/39

(57) ABSTRACT

A device for treating hypo-orgasmia or anorgasmia is described. Energy transfer leads that transfer energy may be implanted proximate to one or more of the dorsal nerves of the clitoris in a female. A signal generator may be used to receive a signal (such as a signal from a handheld remote) to begin the transfer of energy via the two energy transfer leads. The signal generator may then generate pulses either in a predetermined manner or a dynamic manner.

### REFERENCE TO RELATED APPLICATION

This application claims the benefit of U.S. Provisional Application No. 60/987,216, filed Nov. 12, 2007, the entirety of which is hereby incorporated by reference.

### BACKGROUND

Female sexual dysfunction (sometimes termed female sexual arousal disorder) is a very common problem in women that may affect between 33-49% of women between the ages of 18 to 59. Female sexual dysfunction may manifest itself in a variety of ways, including hypoorgasmia (in which the patient has a less than normal ability to achieve orgasm with adequate stimulation) or anorgasmia (in which the patient is unable to achieve orgasm even with adequate stimulation). The incidence of these conditions likely increases with advancing age.

There are several current treatments for female sexual dysfunction. One treatment focuses on psychotherapy in which the female sexual dysfunction is classified as a psychiatric disorder requiring counseling. Another treatment focuses on pharmacologic agents in which the patient is provided with medication, such as anti-depressants. Still another treatment focuses on mechanical external stimulation in order for the patient to achieve orgasm.

Even though these techniques are available, there is still a need to treat sexual dysfunction (such as female sexual dysfunction) that is easier to implement and to operate.

### SUMMARY

The present invention is defined by the attached claims, and nothing in this section should be taken as a limitation on those claims. According to one aspect, a system and method for treatment of hypo-orgasmia and anorgasmia are provided. Energy transfer leads that transfer energy may be implanted proximate to one or more of the dorsal nerves of the clitoris in a female. The dorsal nerves of the clitoris are bilateral and symmetrical in nature. Thus, a unilateral energy transfer lead (proximate to one of the bilateral dorsal nerves) or bilateral transfer leads (proximate to two of the bilateral dorsal nerves) may be used. One example of the energy transfer leads are electrodes that transfer electrical energy. Other types of leads may be used to transfer various types of energy, such as ultrasonic energy, infrared light, magnetic nerve stimulation, chemical energy transfer, or the like.

An internal device, such as a signal generator, may be used to receive a signal to begin the transfer of energy via the two energy transfer leads. The signal may be received from a device external to the body (such as a handheld remote) or may be received from a device internal to the body (such as a sensor within the body that senses a body parameter such as temperature). The internal device may then generate pulses so that at least two of the dorsal nerves are stimulated at the same time. The pulses may be generated in a predetermined manner, such as by using a stored program on the signal generator to generate the electrical pulses. Or the pulses may be dynamically generated. As one example, the signal generator may store a plurality of programs to generate different types of electrical pulses. The handheld remote may send a signal indicative of a selection of one of the stored programs. As another example, the handheld remote may send a signal to the signal generator indicative of increasing or decreasing the energy pulses currently being generated. As still another example, the dynamic energy pulses may be determined based on a signal received from a sensor internal to the female. The sensor may sense a parameter of the female (such as temperature, pressure on the clitoris, etc.), and the signal generator may receive the sensed parameter and select the energy pulses based on the sensed parameter (such as increasing the energy pulses if based on the pressure on the clitoris). The following description will now be described with reference to the attached drawings.

### BRIEF DESCRIPTION OF THE DRAWINGS

FIG. 1 is an illustration of the dorsal nerve of the clitoris and the nerves it originates from.

FIG. 2 is a block diagram of one example of a system for treatment of hypo-orgasmia and/or anorgasmia, including a signal generator and an external device.

FIG. 3 is an expanded block diagram of the signal generator shown in FIG. 2.

FIGS. 4a-d are illustrations of an exemplary surgical procedure.

FIGS. 5a-b are a flow chart for operation of the signal generator depicted in FIG. 3.

### DETAILED DESCRIPTION

As discussed in the background section, many people suffer from sexual dysfunction, including hypo-orgasmia and anorgasmia. There have been several attempts in treatment of hypo-orgasmia and anorgasmia. However, these treatments fail in providing direct stimulation of the dorsal nerves in the female patient. Thus, a method and apparatus is disclosed for the treatment of sexual dysfunction, including hypo-orgasmia and anorgasmia, using a programmable implantable device that includes one or more leads for a direct connection to one or more dorsal nerves in the patient. The method and apparatus may be used either alone or in combination with other methods in the treatment of sexual dysfunction. A method is also disclosed for the surgical procedure to place the implantable device within the patient.

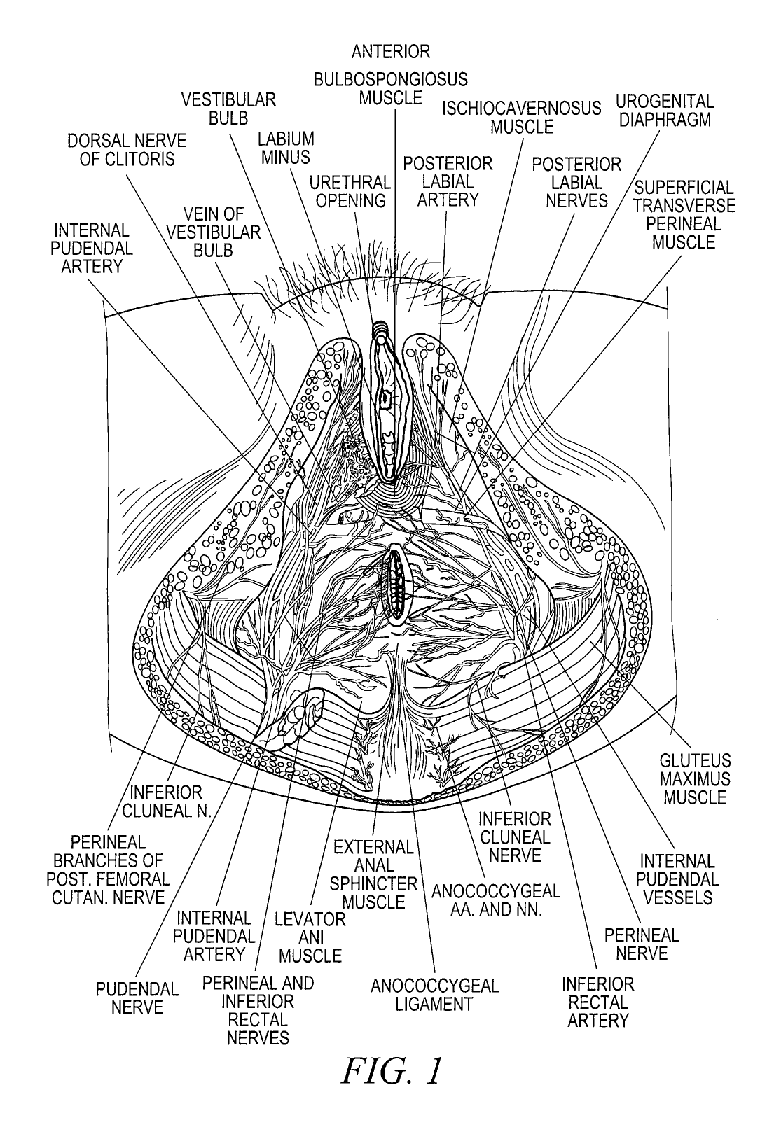

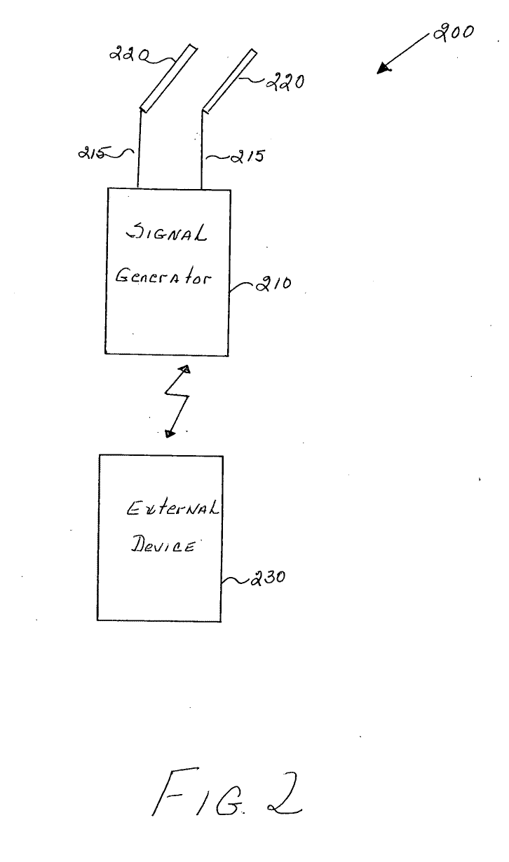

FIG. 1 shows an illustration of the dorsal nerve of the clitoris in a female (highlighted in the upper left portion of the figure) and the nerves it originates from. Though the following discussion focuses on treatment of sexual dysfunction in females, the method and apparatus may likewise be used to treat sexual dysfunction in males. The dorsal nerve of the clitoris comprises terminal branches of the sacral nerve roots to innervate the clitoris. The dorsal nerve of the clitoris is analogous to the dorsal nerve of the penis in males. The dorsal nerve of the clitoris has input into the sacral nerve roots. The clitoral and perineal neurovascular bundles are large, paired terminations of the sacral nerve roots. The clitoral neurovascular bundles ascend along the ischiopubic rami to meet each other and pass along the superior surface of the clitoral body supplying the clitoris. The neural trunks pass largely intact into the glans. These nerves are at least 2 mm in diameter even.

As discussed above, the method and apparatus uses an implantable device in order to provide direct nerve stimulation to the dorsal nerve. The direct nerve stimulation may comprise any form of energy transfer to the dorsal nerve, such as a transfer of electrical energy, ultrasonic energy, infrared light, magnetic nerve stimulation, etc. Also, the direct nerve stimulation may comprise a chemical stimulation through an implantable reservoir. The below discussion focuses on energy transfer to the dorsal nerve using electrical energy. However, any other type of energy transfer may be used.

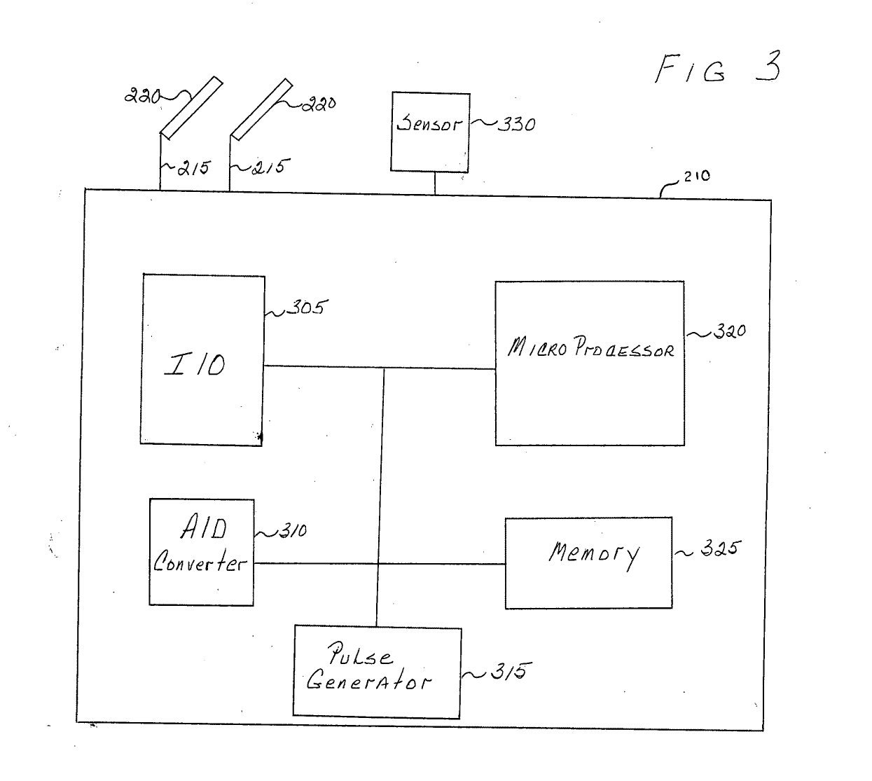

FIG. 2 is a block diagram of one example of a system for treatment of hypo-orgasmia and/or anorgasmia, including a signal generator and an external device. Portions of system 200 may be implanted below the skin of a patient. The system may include generally one or more electrodes 220 implantable in or around one or more nerves of the clitoris (such as around or in the vicinity of the dorsal nerve of the clitoris). One or more electrodes 220 may serve to deliver electrical energy, in the form of electrical stimuli, to the one or more dorsal nerves of the clitoris under the control of the signal generator 210. In addition, though not necessary, the signal generator 210 may use electrodes 220 to receive response field potentials to the electrical stimuli delivered to the dorsal nerve. Alternatively, additional electrodes may be implanted in the dorsal nerve to sense the response field potentials to the electrical stimulation. One example of system 200 may include the Medtronic Activa® Therapy System, aspects of which are described in U.S. Pat. No. 7,006,872. U.S. Pat. No. 7,006,872 is incorporated by reference herein in its entirety. The Medtronic Activa® Therapy System is a nerve stimulator with electrodes implanted in the brain to treat, for example, Parkinson's disease. The signal generator in the Medtronic Activa® Therapy System may be implanted in the clavicle area. Another example of system 200 may include the Medtronic Synergy™ System for the management of neuropathic pain of a non-structural nature. Still another example of system 200 may include Medtronic Itrel®, Synergy™, and Mattrix® Neurostimulation Systems for neurostimulation and site-specific drug delivery as an aid in the management of chronic, intractable pain of the trunk or limbs. Yet another example of system 200 may include the Medtronic InterStim® Therapy System, which is a nerve stimulator for the treatment of incontinence. Still another system may include the Medtronic RESTORE™ Rechargeable Neurostimulation System for treating the patients suffering from chronic pain.

In the example illustrated in FIG. 2, electrodes 220 are coupled to the signal generator 210 through leads 215. Alternatively, electrodes 220 may communicate wirelessly with signal generator 210. Electrodes 220 may take the form of a device capable of detecting nerve cell or axon activity. An external device 230 may act as a programmer and/or controller for the signal generator 210. For example, the external device 230 may comprise a handheld computing device (though a stationary computing device may similarly be used). The external device 230 may be used to communicate and program the signal generator 210. The external device 230 may transmit and receive data from the signal generator 210, communicating with the signal generator 210 through a wireless link. The wireless link may comprise a radio frequency signal, ultrasound signal, infrared signal or other like communication means.

As discussed above, the one or more electrodes 220 on leads 215 may serve to deliver the electrical stimuli. In addition (though not necessary), the one or more electrodes 220 on lead 215 may also receive the response field potentials. Or, additional electrodes on one or more additional leads may be used to either deliver electrical stimuli and/or sense the response field potentials. Depending upon the situation, one or more stimulation/sensing leads with any number of electrodes may be used.

The signal generator 210 may include a pulse generator and a signal analyzer encased in an implantable housing, as discussed in more detail in FIG. 3. The pulse generator may produce and deliver electrical pulses to electrodes 220. The signal analyzer may process signals received by electrodes 220, and determine and/or predict the response of the dorsal nerve to the delivered electrical pulses.

FIG. 3 is a block diagram depicting signal generator 210 in greater detail. Response field potentials sensed with one or more of electrodes 220 may be sent and received via an input/output (I/O) device 305. I/O device 305 may include an amplifier for amplifying signals and a filter for filtering signals. Signals for output on electrodes 220 may be converted from a digital representation to an analog representation via digital to analog converter 310. The response field potentials may be input to a microprocessor 320 for processing.

The microprocessor 320 may be used for processing the response field potentials received by the one or more of electrodes 220 and determine/predict the response of the dorsal nerve to the delivered electrical pulses. A memory may house one or more programs used by the microprocessor 320 to analyze the response field potentials received in order to determine/predict the response of the dorsal nerve to the delivered electrical pulses.

Microprocessor 320 may be coupled to a pulse generator 315. The pulse generator 315 may deliver electrical stimuli to the one or more of the electrodes 220 implanted in the clitoris under the control of microprocessor 320. The pulse generator 315 may be used to deliver stimuli having a pulse pattern, as described in more detail below.

The system 200 may comprise a closed-loop or an open loop system. In a closed loop system (whereby activation of the signal generator 210 is accomplished automatically), the electrodes 220 may sense a predetermined body activity. The predetermined body activity may be used to activate the delivery of the electrical pulses and/or may be used to determine the amount of the electrical pulses after activation, as discussed in more detail below. In particular, one, both or none of activation of the electrical pulses or changes in the electrical pulses may be determined via sensors that sense body activity. For example, the activation of the electrical pulses and subsequent determination of the electrical pulses may be determined by one or more sensors, as discussed in more detail below. As another example, activation may be triggered by an external signal (such as signals generated by external device 230) and the subsequent determination of the electrical pulses may be determined by one or more sensors. As still another example, the activation of the electrical pulses and subsequent determination of the electrical pulses may be determined by one or more external signals (such as signals generated by external device 230).

One example of a predetermined body activity may comprise a predetermined amount of nerve stimulation on the dorsal nerve (which may be sensed by the one or more electrodes 220). Alternatively, another sensor may be used to sense the predetermined body activity. For example, a pressure sensor may be used to sense if a predetermined section of the body is receiving pressure (such as pressure on the clitoris). The output of the pressure sensor may be input to the microprocessor 320 to activate delivery of electrical pulses and/or to determine/change the electrical pulses (such as the microprocessor 320 interpreting a greater amount of pressure or a greater frequency of pressure as an indication to increase the amplitude of the electrical pulses, as discussed in more detail below). Other sensors may sense various other body activities, such as temperature changes (which may be sensed via a temperature sensor), moisture changes, or molecular changes (which may be sensed via a pH sensor or ion gradient sensor).

This predetermined body activity may be indicative of an attempt to stimulate the clitoris. If sensed, microprocessor 320 may control delivery of electrical pulses (via the pulse generator 315) to the dorsal nerve. The predetermined amount of nerve stimulation indicative of an attempt at stimulation of the clitoris may be a self-learning process or may be an iterative process based on input from the patient. Alternatively, the microprocessor 320 in the signal generator 210 may receive a signal external to the patient (such as via external device 230) to begin delivery of electrical pulses. For example, the external device 230 may have an “activate” switch which activates the delivery of the electrical pulses via the pulse generator 315.

At the time of implantation of the signal generator 210 within the patient, the clinician may program certain key parameters into the memory of the implanted device or may do so via telemetry. These parameters may be updated subsequently as needed. Alternatively, the clinician may elect to use default values. The clinician may adjust the parameters of the electrical pulses via telemetry with a medical device programmer.

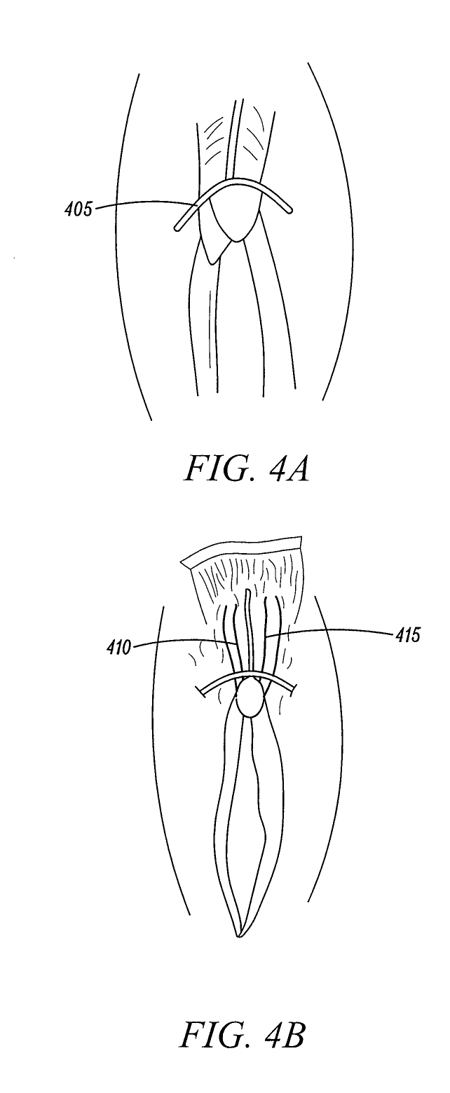

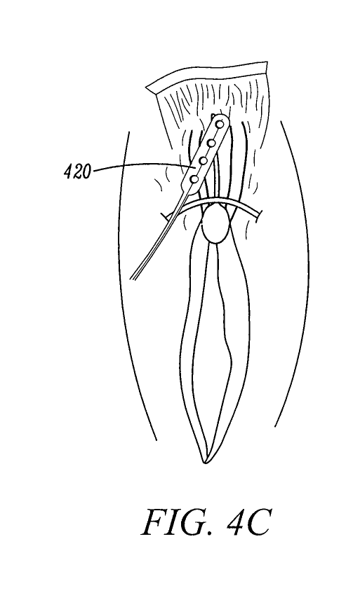

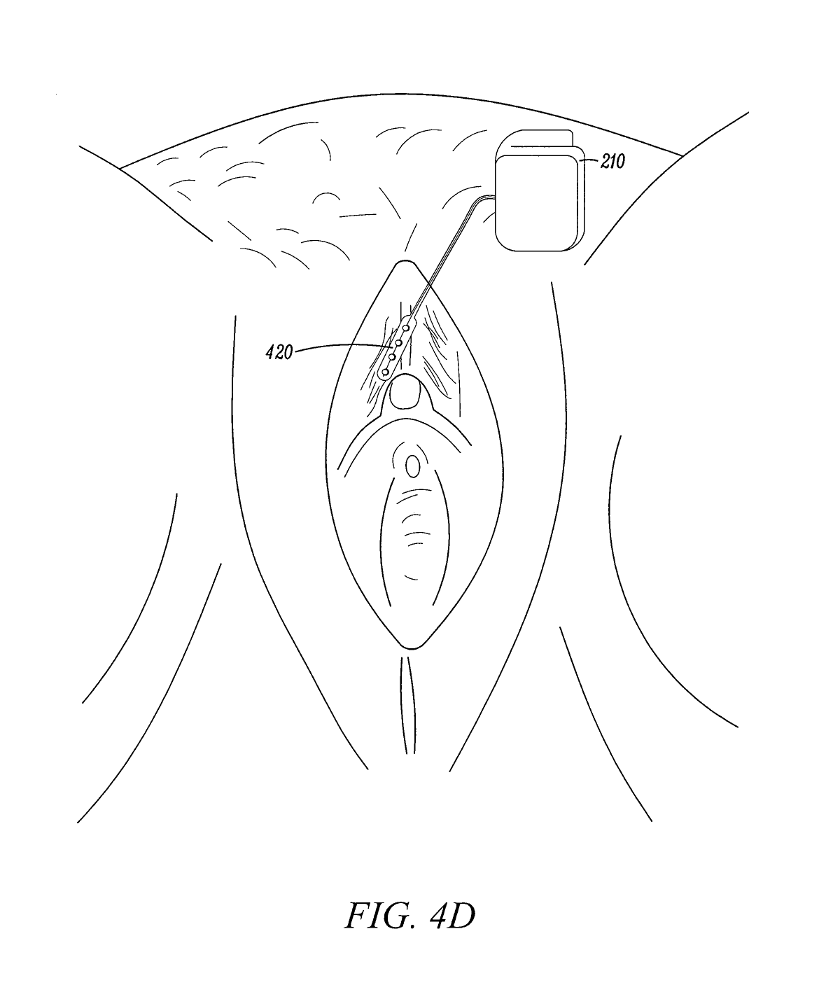

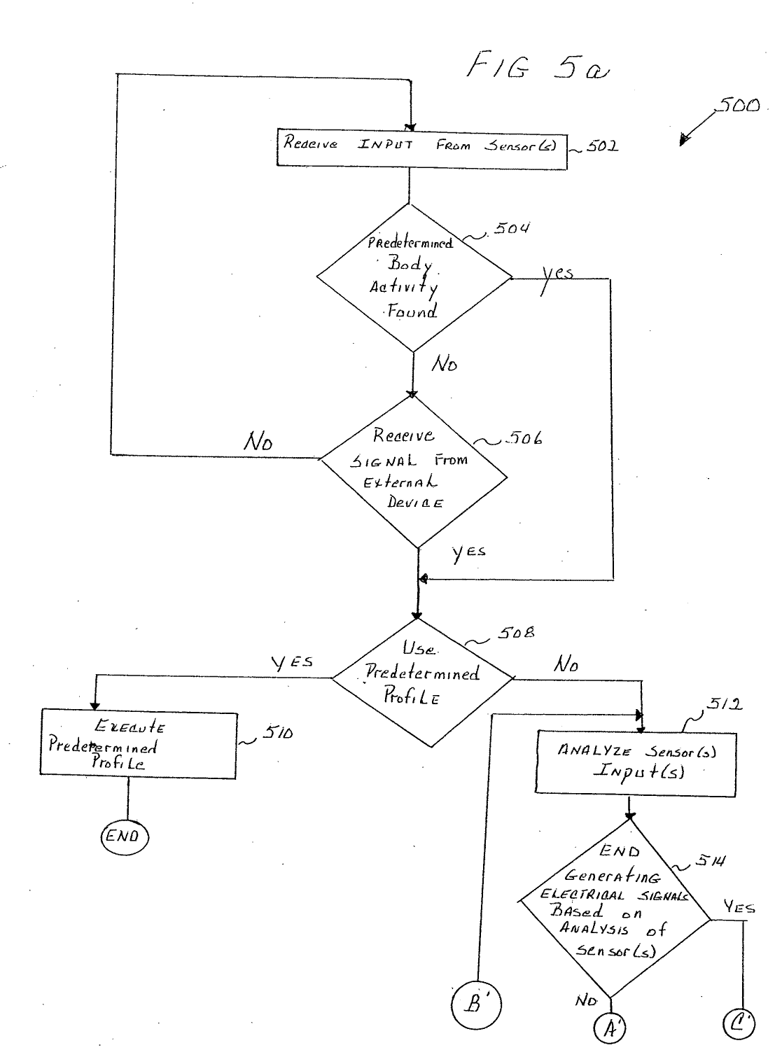

FIGS. 4a-d are illustrations of an exemplary surgical procedure. One or both clitoral nerves may be approached from an incision in the superior fold of the hood of the clitoris. For example, a small transverse incision may be made in the clitoral hood, as indicated by element 405 in FIG. 4a. Alternatively, the leads may be placed percutaneously on one or both sides of the nerves with a large bore needle rather than making an incision around the clitoral hood.

Surgical dissection using fine scissors may be carried out through the incision in the sub-mucosal plane over the dorsal vein of the clitoris which may be clearly visible. The dissection may be carried towards the suspensory ligament attachment to the pubic bone (i.e., dissection may be continued over the dorsum of the clitoris until the dorsal neurovascular bundle is exposed towards pubic symphisis). The two dorsal nerves of the clitoris (identified as elements 410 and 415 in FIG. 4b) may be identified on either side of the dorsal vein. The diameter of the dorsal nerves starts increasing about one centimeter behind the glans of the clitoris, and at any location from this point to the level of the suspensory ligament, the nerve stimulator leads may be placed. In particular, the leads may be placed proximate to the surface (such as from 1 to several millimeters from) one or more of the dorsal nerves (such as proximate to one or both of the paired structures of the bilateral dorsal nerves). The lead is identified as element 420 in FIG. 4c.

Lead placement may depend on the type of leads used and can be placed in variety of ways. As one example, the leads may be placed on the surface of the nerve(s) and secured in place using fine sutures (e.g., 9-0). As another example, the leads may be placed around the nerve as a cuff by elevating the nerve(s) along its(their) course over the dorsum of the clitoris (3-7 cm length).

The other end of the lead(s) may be tunneled in a subcutaneous plane to the location of the signal generator 210. Placement of the signal generator 210 may be anywhere in the body. Convenient locations may be the subcutaneous fat of the mons pubis, the subcutaneous fat in the lower abdomen or the subcutaneous fat in the hips. Custom or existing tunneling instruments may be used for threading the lead wires in each location (such as a flexible metal 2-3 mm diameter blunt hollow needle of appropriate length).

For example, the signal generator may be placed in the mons fat pad if there is adequate overlying fat or may be placed in the lower abdomen through a separate incision in the mucosa above the hood of the clitoris and below the mons pubis. The dissection for the signal generator pocket may extend all the way to the lower abdomen through this incision. If the patient does not have adequate fat tissue to hide the signal generator, the “love handle” or gluteal area may be used. An incision in the upper part of the gluteal crease may be used in order to avoid any visible scars.

After creating a tunnel towards the location in which the signal generator will reside (such as the mons pubis fat pad), the electrode ends may be brought externally in order to ensure that the placement of the lead is effective in producing an orgasm. For example, prior to the permanent implantation of the signal generator, the leads may be brought through the skin and a “test” may be performed during the first few days following the placement of the leads. Once complete functionality is verified the signal generator may be implanted anywhere in the body as a separate procedure. If the percent of patients who benefit from this device is high, then both leads and signal generator may be implanted in the same procedure.

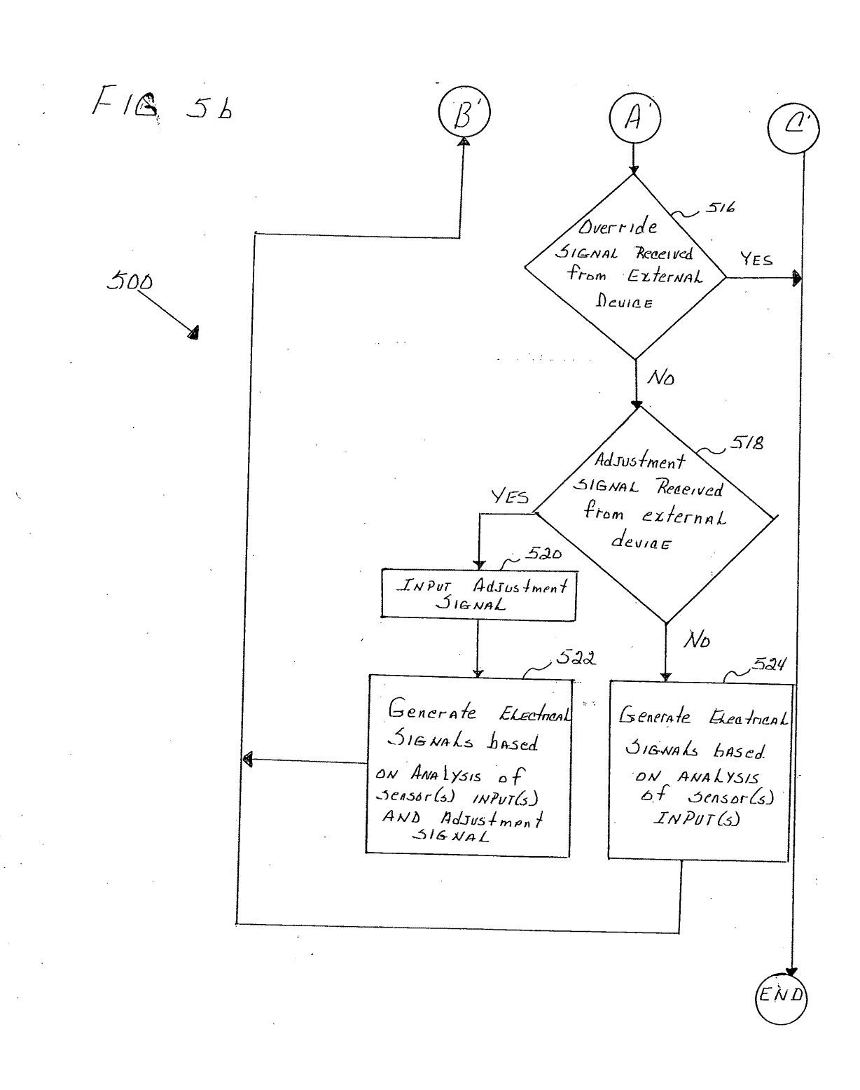

FIGS. 5a-b are a flow chart 500 for operation of the signal generator depicted in FIG. 3. As discussed above, one, both or none of activation of the electrical pulses or changes in the electrical pulses may be determined via sensors that sense body activity. As shown at block 502, input may be received from one or more sensors. The sensors may sense any aspect of body activity, and may include electrical sensors, temperature sensors, pH sensors, etc. Based on the sensor input, a predetermined body activity is analyzed. The analysis of the sensor input may comprise analyzing one reading of the sensor(s) (such as a single reading of the temperature sensor), or may comprise analyzing multiple readings of the sensor(s) (such as analyzing a history of temperature sensor readings to determine if there is an upward trend in the temperature). The analysis of the sensor readings may determine if a predetermined body activity is found, as shown at block 504. If it is found, the electrical signals may be generated, as discussed in more detail below. If the predetermined body activity is not found, it is determined whether a signal has been received from the external device, as shown at block 506. As discussed above, the external device may send a signal to activate the signal generator. In this way, the patient may control activation using the external device.

Once it is determined to activate the signal generator, it may then be determined whether to use a predetermined profile, as shown at block 508. The predetermined profile may be pre-programmed into the signal generator and may simulate the pattern of electrical stimulations to achieve orgasm. The predetermined profile may comprise a known existing waveform or may comprise a waveform tailored to the specific patient. If it is determined to use a predetermined profile, the predetermined profile is executed, as shown at block 510. Alternatively, selection of the predetermined profile may be based on the patient's input. A predetermined profile, selected from several predetermined profiles stored in the signal generator, may be selected based on input sent from the patient via the external device.

If it is determined not to use a predetermined profile, one or more inputs from one or more sensors are analyzed, as shown at block 512. For example, inputs from a single sensor or multiple sensor may be used in the analysis. And, a single input or multiple inputs (such as a history of inputs) may likewise be used in the analysis. The analysis of the input(s) may comprise using a look-up table to determine what electrical signals should be generated based on the input(s). For example, an input may be correlated to a predetermined output signal.

The analysis of the sensor input(s) may indicate that the electrical signals should be ended, as shown at block 514. For example, if the sensor input(s) are outside of a predetermined range, the signal generator may determine to end generating electrical signals. Further, it is determined if a signal is received from the external device to end generating electrical signals, as shown at block 516. This enables the patient to control ending the electrical signals using the external device.

The signal generator also determines whether an adjustment signal has been received from the external device, as shown at block 518. The patient may send a signal to the signal generator indicating a request to change the electrical pulses generated. For example, the external device may include an “up” arrow switch and a “down” arrow switch. The patient may press the “up” arrow switch, thereby triggering the external device to send a signal to the signal generator indicating that the patient seeks to increase the electrical signals generated. If an adjustment signal is received, it is input (as shown at block 520). Further, the adjustment signal is used, along with the analysis of the sensor(s) input(s) in order to generate the electrical signals, as shown at block 522. If an adjustment signal is not received, the electrical signals are generated based on the analysis of the sensor(s) inputs(s), as shown at block 524. As discussed above, the analysis may comprise using a look-up table, whereby sensor inputs are correlated to certain electrical signals. Once the electrical signals are determined, the signal generator may generate the electrical signals for transmission to the electrodes.

It is intended that the foregoing detailed description be understood as an illustration of selected forms that the invention can take, and not as a definition of the invention. It is only the following claims, including all equivalents, that are intended to define the scope of this invention.

What is claimed is:

1. A method for treating a female with sexual dysfunction comprising:
providing at least one energy transfer lead for transferring energy;
implanting the at least one energy transfer lead proximate to at least one dorsal nerve of the clitoris in order to transfer energy to the at least one dorsal nerve;
using at least one device internal to the female to receive at least one signal in order to begin transfer of energy via the energy transfer lead;
generating energy pulses via the at least one device in accordance with at least one stimulation parameter; and
delivering the energy pulses to at least the at least one dorsal nerve of the clitoris adjacent to the at least one energy transfer lead.

2. The method of claim 1, wherein at least one energy transfer lead for transferring energy comprises providing at least two electrical leads;
wherein implanting the at least one energy transfer lead proximate to at least one dorsal nerve of the clitoris in order to transfer energy to the at least one dorsal nerve comprises implanting the at least two energy transfer leads proximate to at least two dorsal nerves of the clitoris in order to transfer energy to the two dorsal nerves; and
wherein the energy pulses comprise electrical pulses.

3. The method of claim 1, wherein the at least one device internal to the female receives the signal from a device external to the female in order to begin the transfer of energy.

4. The method of claim 1, wherein the at least one device internal to the female receives the signal from a sensor internal to the female in order to begin the transfer of energy.

5. The method of claim 4, wherein the sensor senses at least one parameter of the female.

6. The method of claim 5, wherein the parameter is selected from the group consisting of temperature, pH, and electrical activity.

7. The method of claim 1, wherein the at least one stimulation parameter for generating the energy pulses comprises a program for generating predetermined energy pulses.

8. The method of claim 1, wherein the at least one stimulation parameter for generating the energy pulses comprises a program for generating dynamic energy pulses.

9. The method of claim 8, wherein the dynamic energy pulses are determined based on at least one signal received from a device external to the female.

10. The method of claim 9, wherein the device external to the female comprises a handheld remote control.

11. The method of claim 10, wherein the device internal to the female stores a plurality of energy pulse programs;
wherein the at least one signal received from the handheld remote control comprises a signal indicative of one of the plurality of energy pulse programs; and
wherein the device internal to the female selects one of the plurality of energy pulse programs based on the signal received from the handheld remote control.

12. The method of claim 10, wherein the at least one signal received from the handheld remote control comprises a signal indicative of increasing or decreasing the energy pulses.

13. The method of claim 8, wherein the dynamic energy pulses are determined based on at least one signal received from a sensor internal to the female.

14. The method of claim 13, wherein the sensor senses at least one parameter of the female;
wherein the at least one device internal to the female receives the sensed parameter; and
wherein the at least one device internal to the female determines the energy pulses based on the sensed parameter.

15. The method of claim 2, wherein the at least two energy transfer leads have a size and shape suitable for placement proximate to the dorsal nerves of the clitoris.
